(19) United States
(12) Patent Application Publication
(10) Pub. No.: US 2024/0200026 A1
(43) Pub. Date: Jun. 20, 2024

(54) COMPOSITIONS, SYSTEMS, AND METHODS RELATED TO PLANT BIOPRINTING

(71) Applicant: North Carolina State University, Raleigh, NC (US)

(72) Inventors: Rosangela Sozzani, Raleigh, NC (US); Timothy J. Horn, Raleigh, NC (US)

(21) Appl. No.: 17/907,076

(22) PCT Filed: Mar. 22, 2021
(86) PCT No.: PCT/US2021/023398

§ 371 Date: Sep. 22, 2022

Related U.S. Application Data
(63) Continuation of application No. 62/993,157, filed on Mar. 23, 2020.

(51) Int. Cl. C12N 5/04; B33Y 10/00; B33Y 30/00; B33Y 80/00

(52) U.S. Cl. CPC C12N 5/04; B33Y 10/00; B33Y 30/00; B33Y 80/00; C12N 2501/10; C12N 2501/30; C12N 2513/00

(57) ABSTRACT

The present disclosure provides materials and methods relating to bioprinting. In particular, the present disclosure provides compositions, systems, and methods for 3D bioprinting plant cells according to pre-determined spatiotemporal patterns to facilitate the high-throughput regeneration of plants having reduced variability.

### CROSS REFERENCE TO RELATED APPLICATIONS

This application claims priority to and the benefit of U.S. Provisional Patent Application No. 62/993,157 filed Mar. 23, 2020, which is incorporated herein by reference in its entirety for all purposes.

### GOVERNMENT SUPPORT

This invention was made with government support under grant number MCB2039285 awarded by National Science Foundation. The government has certain rights in this invention.

### FIELD

The present disclosure provides materials and methods relating to bioprinting. In particular, the present disclosure provides compositions, systems, and methods for 3D bioprinting plant cells according to pre-determined spatiotemporal patterns to facilitate the high-throughput regeneration of plants having reduced variability.

### BACKGROUND

Breeding efforts have struggled to cope with the urgent need of feeding the increasing world population, but protoplast transformation methods (e.g., fusion, editing, or mutagenesis) have the potential to enable easy genetic manipulation and crop improvement. To make a significant contribution towards providing crop genetic variability and technological advances, it is essential to have systems that are efficient and reliable to regenerate whole plants from cells/protoplasts. If plant regeneration using spatiotemporal arrangement of protoplasts is to become available, the attributes of native tissues can be determined at temporal as well spatial length-scales, ranging from sub-micron (e.g., interfaces of adjacent cells, cells biomaterials, etc.) to macro (e.g., tissue- and organ-specific 3D geometry).

Currently, the generation of plants with traits of interest requires expensive and time-consuming hybridization, genetic research, and generations of successive plantings. The use of somatic embryogenesis has great potential for in vitro propagation and improvement of useful plants, but current methodologies are slow, costly, and often unreliable. Significant advances are needed for plant breeding pipelines to effectively and sustainably increase crop production. The ability to bioprint tissues and organs as well as other functional cellular and biomolecular structures for therapeutic, diagnostic, and research applications is revolutionizing the medical field, but has not yet been applied to the agricultural field.

### SUMMARY

Embodiments of the present disclosure include a matrix comprising a plurality of bioprinted plant cells. In accordance with these embodiments, the plurality of bioprinted plant cells are deposited in the matrix according to a pre-determined spatial pattern.

In some embodiments, the matrix is three-dimensional (3D) or two-dimensional (2D). In some embodiments, the matrix comprises at least one of agar, hydrogel, nanofiber, plant biomaterials, and any combinations thereof.

In some embodiments, the plurality of plant cells includes at least one plant cell obtained from a meristematic region of a root and/or at least one plant stem cell. In some embodiments, the plurality of plant cells includes at least one stem cell obtained from a shoot and/or root stem cell niche. In some embodiments, the plurality of plant cells includes at least one cell obtained from a shoot and/or root apical meristem of a plant species.

In some embodiments, the plurality of plant cells are deposited in the matrix according to a pre-determined temporal pattern. In some embodiments, the plurality of plant cells are contained within a bioink composition, and deposited into the matrix in the bioink composition. In some embodiments, the matrix further includes at least one of a hormone(s), a phytohormone(s), a nutrient(s), an antibiotic(s), a prebiotic(s), a probiotic(s), a peptide(s), a polypeptide(s), a protein(s), a other growth factor(s), and any combinations thereof. In some embodiments, the pre-determined spatial pattern induces the plurality of plant cells to form a callus.

In some embodiments, at least one of the plurality of plant cells is naturally occurring. In some embodiments, at least one of the plurality of plant cells is genetically modified.

In some embodiments, the plurality of plant cells are deposited in the matrix according to at least one additional pre-determined metric selected from cell number, cell density, cell-type, cell arrangement, and bioink composition.

In some embodiments, the plurality of plant cells are deposited using a bioprinting device.

Embodiments of the present disclosure also include a plant callus formed from any of the plurality of bioprinted plant cells described herein.

Embodiments of the present disclosure also include a plant organ, organoid, or tissue formed or derived from any of the bioprinted plant cells described herein.

Embodiments of the present disclosure also include a system for bioprinting plant cells. In accordance with these embodiments, the system includes a plurality of plant cells, a matrix whereupon the plurality of plant cells are deposited, and a bioprinting device.

In some embodiments, the bioprinting device deposits the plurality of plant cells in the matrix according to a set of pre-determined instructions. In some embodiments, the bioprinting device includes a processor component and a software component. In some embodiments, the software component includes the set of pre-determined instructions for bioprinting the plurality of plant cells. In some embodiments, the processor component executes the set of pre-determined instructions.

In some embodiments, the set of pre-determined instructions includes spatial and temporal information for depositing the plurality of plant cells in the matrix. In some embodiments, the spatial and temporal information is based on a computational model that includes gene expression data, cell number, cell-type, rate of cell division, and any combinations thereof.

Embodiments of the present disclosure also include a method of producing a plant callus. In accordance with these embodiments, the method includes bioprinting a plurality of plant cells in a matrix, and culturing the bioprinted plant cells in the matrix to induce the formation of a callus. Embodiments of the present disclosure also include a plant callus formed using these methods, as well as a plant organ, organoid, or tissue, as described further herein.

### BRIEF DESCRIPTION OF THE DRAWINGS

FIG. 1: Representative confocal images of control and bioprinted cells at 6, 12, 24, and 48 hours (left), percent viability (top right) and cell wall formation (bottom right). Cells are stained in green with FDA for viability, cells are stained in blue with calcofluor white for cell wall formation, and red cells are expressing pSCR-SCRmCherry, as fluorescent marker used for CEI and endodermal cell identity.

FIGS. 2A-2C: Representative model predicting cell identity depending on the position of the cell (top) and the number of each cell-type (bottom). A vascular initial (VASC) placed at the position of a columella stem cell (CSC) changes cell identity (top). Only in the presence of additional quiescent cells (QCs) is it predicted that VASC are maintained (bottom) (A). Representative 3D PCA of stem cell transcriptional profiles. Non-stem cells (NSCs); Stem cell niche (SCN); Cortex endodermis initial (CEI); Protophloem (Protophlo); Epidermis/lateral root cap (Epi/LRC); Xylem (Xyl) (B). Distribution of the selected stem cell-enriched genes showing expression across and specific to each stem cell (C).

FIG. 3: Representative model of single cell gene expression profile analysis, cellular auxin levels, cell rearrangements, cell positions, and predicted gene regulatory network architectures, which serves as input data for machine learning models, as provided herein.

FIG. 4: Representative model of the production of 3D bioprinting plant cells according to pre-determined spatial patterns to facilitate the high-throughput regeneration of plants having reduced variability.

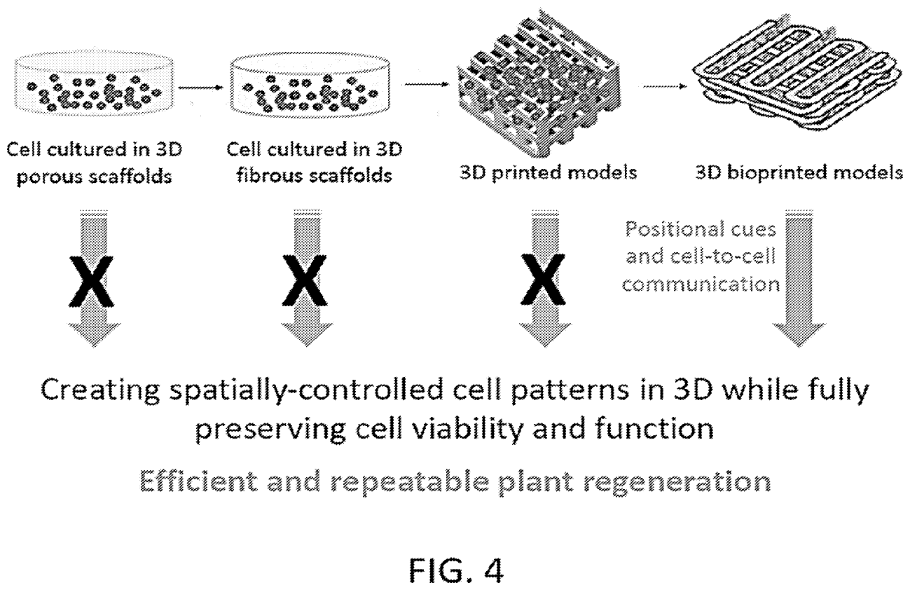

FIG. 5: Representative flowchart showing stem cell selection and bioprinting of plant cells into a platform: i) Plantlets of Arabidopsis—the stem cell populations are located at the tip of shoots and roots (box) and provide a continuous supply of cells: ii) Confocal image of a specific root stem cell-type labeled with a fluorescent marker. Note: marker lines for different stem cells exist: iii) Fluorescent Activated Cell Sorting (FACS) of cells labeled with fluorescent markers. RNA for next generation sequencing (RNA-seq) was extracted and amplified from only a few stem cells (˜50 pg of RNA). iv) 3D Bioplotter machine into which cells are fed and maintained in a sterile environment for deposition. v) Close up view of the Bioplotter needle depositing live plant cells.

FIGS. 6A-6B: (A) A representative image of cell clusters at 5 days after bioprinting taken with the confocal microscope Zeiss 880. Cells were stained with fluorescein diacetate (FDA) and calcofluor white to identify viable cells and the formation of cell walls, respectively. (B) Protoplasts were isolated from pSCR:SCR-mCherry meristematic root cells, 3D bioprinted with the CELLINK BIOX, and imaged immediately after bioprinting (day 0) and 1, 3, 5, 7, and 9 days after bioprinting with the confocal microscope Zeiss 880. Cell viability was evaluated with fluorescein diacetate (FDA) staining. The protoplasts were bioprinted in PIM with 0.6% low melting agar. n=3-6 images with 50-200 cells per image. Error bars represent standard error.

FIGS. 7A-7C: Cell viability of manual pipetted and 3D bioprinted protoplasts isolated from meristematic root cells is comparable. (A-B) Percentage cell viability achieved with the 3D bioplotter (Envisiontec) (A) and the CELLINK BIOX bioprinter (B). The percentage of viable cells is represented relative to the 0 time point (i.e. immediately after manual pipetting (grey bars, control)) or 3D bioprinting (green bars, 3D bioprinted). (C) Cell viability was evaluated with fluorescein diacetate (FDA) staining using the confocal microscope Zeiss 880. The protoplasts were bioprinted in PIM with 0.6% low melting agar. n=2-6 images with 50-200 cells per image. Error bars represent standard error.

FIGS. 8A-8C: Cell viability over time of 3D bioprinted protoplasts. (A-C) Protoplasts were isolated from Col-0 meristematic (A) and differentiated (B) root cells and from SCR:SCR-mCherry meristematic root cells (C), 3D bioprinted with the CELLINK BIOX, and imaged immediately after bioprinting (day 0) and 1, 3, 5, 7, 9, and 11 days after bioprinting with the confocal microscope Zeiss 880. Cell viability was evaluated with fluorescein diacetate (FDA) staining. The protoplasts were bioprinted in PIM with 0.6% low melting agar. n=3-6 images with 50-200 cells per image. Error bars represent standard error.

FIGS. 9A-9B: Total number of protoplasts over time to identify cell divisions. (A-B) Distribution of the total number of protoplasts isolated from Col-0 meristematic (A) and differentiated (B) root cells. The protoplasts of the same 3D bioprinted structure were imaged with the confocal microscope Zeiss 880 immediately after bioprinting (day 0) and 1, 4, and 5 days after bioprinting. An increase in the number of protoplasts points towards the generation of additional cells through cell division. The protoplasts were bioprinted in PIM with 0.6% low melting agar. n=5-7 images with 30-150 cells per image.

FIGS. 10A-10C: Cell identity of ground tissue is retained for at least 3 days after bioprinting. (A-B) Protoplasts were isolated from J0571 meristematic (A) and differentiated (B) root cells. The protoplasts of the same 3D bioprinted structure were imaged with the confocal microscope Zeiss 880 immediately after bioprinting (day 0) and 1, 3, and 5 days after bioprinting. (C) Representative image of protoplasts expressing GFP 3 days after bioprinting. The protoplasts were bioprinted in PIM with 0.6% low melting agar. n=5-7 images with 30-150 cells per image.

FIG. 11: Representative data shows that cell viability is not affected by cell density. Protoplast densities and cell viability of the experiments from FIG. 8 were plotted in a scatterplot.

FIGS. 12A-12D: Demonstration of encapsulation of multiple cell types within different hydrogels. (A) Encapsulated Col-0 Arabidopsis thaliana protoplasts and J0571 protoplasts in 1% (w/v) Sodium Alginate hydrogel crosslinked with 2 mM Calcium chloride solution after multiple days of culturing. (B) Encapsulated tobacco BY-2 cells in 1% (w/v) Sodium Alginate hydrogel crosslinked with 2 mM Calcium chloride solution after multiple days of culturing. (C) Encapsulated tobacco BY-2 cells in 0.5% (w/v) Sodium Alginate hydrogel crosslinked with 2 mM Calcium chloride solution.

FIG. 13: Representative schematic of a workflow for generating 3D bioprinted plant microcalli, according to a general protocol developed for Arabidopsis.

### DETAILED DESCRIPTION

Human existence depends on food, fiber, shelter, and fuel from plants. In order to meet the demands of a growing population, it is important to effectively and sustainably increase crop production, which requires game-changing advances in plant design. Regulation of plant growth and biomass is dependent on the continuous generation of stem cells that differentiate to form all the cells, tissues, and organs of the plant. Understanding the spatial and temporal control of stem cell self-renewal and their differentiation is critical for generating predictable outcomes and redesigning plants for future needs. The Arabidopsis root tip contains a number of stem cell populations that divide to form two daughter cells. One of the daughter cells becomes the new stem cell, while the other daughter cell differentiates into specific tissues. The stem cells are confined in a region of the root tip called the stem cell niche (SCN), which contains Quiescent Center (QC) cells, a group of relatively mitotically inactive⁴ cells known to provide stem cell maintenance signals. The three stem cells adjacent to the QC divide shootwards to form the lateral and proximal root tissues and include: the lateral root cap/epidermis initials (LRC/EPI), the cortex and endodermis initials (CEI), and the vascular initials (VASC). Distal to the QCs are the columella stem cells (CSCs). Maintenance and differentiation of cells within the SCN is coordinated through cell-to-cell signaling mechanisms that incorporate small molecules and/or transcription factors as signaling molecules. Moreover, when the root tip is excised, including the region of the QC and all the stem cell initials, many of the remaining proliferating cells within the root give rise to new stem cells, creating a new SCN⁵. These cells, thus, form new cell types distinct from their original cell fate, exemplifying the potential of plant cells to respond to local signals (e.g., signals within the local microenvironment such as auxin and mobile transcription factors).

To study cell-to-cell communication, including local signals and cell interactions, driving these developmental processes, it is imperative to manipulate individual cells within their microenvironment. In mammalian studies, 3D bioprinting technologies have been applied for tissue engineering, efficient screening, and personalized treatment. 3D bioprinting allows for the precise, high-throughput deposition of multiple cell types, biomaterials, and growth factors simultaneously at a high resolution. Just as with conventional 3D printing, there are multiple methodologies that can be used to perform 3D bioprinting and recapitulate specific geometrical arrangements, including inkjet, extrusion, laser-assisted, photopolymerization, and scaffold-free spheroid-based bioprinting. Specific arrangements are made possible based on methodology selection, such as continuous printing, drop-on-demand printing, and even single-cell printing. The ability to 3D bioprint tissues and organs as well as other functional cellular/biomaterial structures for therapeutic, diagnostic, and research applications is already revolutionizing the medical field. For example, human-induced pluripotent stem cells have been bioprinted to study cell fate, phenotypic variation, and tissue regeneration. Although 3D bioprinting technology is particularly well suited to generate layer-by-layer, micro-structured geometries and could be used to precisely pattern the plant multi-layer stem cell niche, this technology has so far been limited to animal cells.

Thus, the field of 3D bioprinting has been completely untapped for plant science and offers a prime opportunity for the fabrication of plant tissues. Nonetheless, while bioprinting either plant or animal tissues, it is imperative to quantitatively measure the attributes of native tissues at length-scales ranging from sub-micron (e.g., interfaces of adjacent cells, biomaterials, etc.) to macro (e.g., tissue- and organ-specific 3D geometry). Consequently, by combining 3D bioprinting with quantitative image analysis and single-cell transcriptomics, it is possible to understand the key factors driving cell identity and differentiation and overall system robustness, potentially driving the design and engineering of plants with new and desired traits.

Section headings as used in this section and the entire disclosure herein are merely for organizational purposes and are not intended to be limiting.

### Definitions.

Unless otherwise defined, all technical and scientific terms used herein have the same meaning as commonly understood by one of ordinary skill in the art. In case of conflict, the present document, including definitions, will control. Preferred methods and materials are described below; although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having.” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising.” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

As used herein, “bioink” or “bioink composition” refers to a composition that is suitable for bioprinting, as described herein. For example, the bioink can be a solution, suspension, gel, or concentrate containing material to be bioprinted (e.g., plant cells or protoplasm that is 3D bioprinted). In some embodiments, the bioink comprises a plurality of cells/protoplasts and as well as other components for supporting the viability, growth, and development of the cells/protoplasts. In some embodiments, the bioink includes at least one of a hormone(s), a phytohormone(s), a nutrient(s), an antibiotic(s), a prebiotic(s), a probiotic(s), a peptide(s), a polypeptide(s), a protein(s), a other growth factor(s), and any combinations thereof. In some embodiments, the bioink includes a carrier or other component that supports deposition into a matrix (e.g., hydrogel). The bioink can be used for bioprinting to obtain a planar and/or sheet-like structure having pre-determined dimensions (3D structure). The planar and/or sheet-like structure can be further deposited to form a 3D construct having a pre-determined shape and structure. Cells in the bioink compositions can exhibit physiological activity before, during, and/or after bioprinting. Additionally, the number of cells that are bioprinted can vary depending on a variety of factors, as would be recognized by one of ordinary skill in the art based on the present disclosure. For example, initial concentrations of cells that are 3D bioprinted can be at least 1500 cells/μl of bioink composition.

As used herein, “bioprint” refers to printing using a material comprising biological substances, including biological molecules derived from biological sources (e.g., proteins, lipids, carbohydrates, nucleic acids, metabolites, and/or small molecules), cells, protoplast, subcellular structures (e.g. organelles, membranes, etc.), groups of cells, groups of subcellular structures, or molecules that are related to biological molecules (e.g., synthetic biological molecules or synthetic analogs of biological molecules). “Printing” refers to a process of depositing a material according to a pre-determined pattern, design or scheme (e.g., spatiotemporal arrangement). “Printing” (such as bioprinting) described herein can be carried out by a variety of methods, including, but not limited to, printing using a printer (such as a 3D printer or bioprinter), printing using an automated or non-automated mechanical process rather than a printer, and printing by manual deposition (e.g. using a pipette).

As used herein, “matrix” generally refers to a structure having a plurality of layers, each layer comprising a cross-linked polymer network that supports the deposition of biological material. In some embodiments, the matrix is three-dimensional (3D) or two-dimensional (2D). In some embodiments, the matrix comprises at least one of agar, hydrogel, nanofiber, plant biomaterials, and any combinations thereof. Additionally, in one example, the matrix material and/or the matrix composition may comprise a gel for bioprinting applications, which may exhibit a rapid transition from a low viscosity solution to a solid-like gel, and which may be seen by an initial increase in shear elastic modulus. Rapid, controllable gelation may enhance printed structure fidelity by minimizing or obviating swelling and dissociation typical of slow gelation processes. The term “gel” may refer to a semi-solid substance that may comprise a gelling agent to provide viscosity or stiffness. The gel may be formed upon use of a gelling agent, such as a thickening agent, crosslinking agent or a polymerization agent, and may comprise a cross-linked structure or a non-cross-linked structure. The gel may be hydrophobic or hydrophilic. Some examples of suitable gels include a hydrogel, thermo-reversible gel, a photo-sensitive gel, a pH sensitive gel, a peptide gel, or a cell type specific gel. Additional examples of gels include silica gel, silicone gel, aloe vera gel, agarose gel, nafion, polyurethane, elastomers (thermoplastic, mineral-oil thermoplastic, etc.), ion-exchange beads, organogels, xerogels and hydrocolloids. Hydrogels include those derived from collagen, hyaluronate, fibrin, alginate, agarose, Pluronic F127, Pluronic F123 (and other poloxamers), chitosan, gelatin, matrigel, glycosaminoglycans, and combinations thereof. In one example, the gel may comprise gelatin methacrylate (GelMA), which is denatured collagen that is modified with photopolymerizable methacrylate (MA) groups. Suitable hydrogels may comprise a synthetic polymer. In certain embodiments, hydrogels may include those derived from poly(acrylic acid) and derivatives thereof, poly(ethylene oxide) and copolymers thereof, poly(vinyl alcohol), polyphosphazene, and combinations thereof. The extracellular matrix material and/or the extracellular matrix composition may comprise a naturally derived such as biocompatible material, one or more extracellular matrix components, including collagen (e.g., I, III, and IV), fibrin, fibronectin, fibrinogen, gelatin (e.g., low and high bloom gelatin and/or temperature treated), laminin, hyaluronates (e.g., hyaluronic acid), elastin, and/or proteoglycans. Other suitable biocompatible materials for the extracellular matrix material and/or the extracellular matrix composition may include variations and/or combinations of cellulose, Matrigel, acrylates, acrylamides, polylactic co-glycolic acid, epoxies, aldehydes, ureas, alcohols, polyesters, silk, carbopol, proteins, glycosaminoglycans, carbohydrates, minerals, salts, clays, hydroxyapatite, and/or calcium phosphate. Further examples may include variations and/or combinations of N-Isopropylacrylamide (NIPAAM), Polyethylene glycol (PEG), gelatin methacrylate (GelMA), Polyhydroxyethylmethacrylate (PHEMA). Combinations of the above listed materials are also contemplated for use as the extracellular matrix material and/or the extracellular matrix composition.

As used herein, “tissue” refers to an ensemble of one or more groups of cells each having the same or similar morphology and functions. Tissue typically further comprises non-cell materials known as intercellular substance, such as extracellular matrix and fibers. A tissue may include a single type of cells or multiple types of cells. As used herein, “organ” refers to a structural unit comprising one or more tissues for serving one or more specific bodily functions. In some embodiments, an organ consists of a single tissue. In some embodiments, an organ comprises multiple tissues. “Artificial tissue” refers to a tissue that is not formed through natural tissue generation or development processes inside a biological organism. In some embodiments, an artificial tissue is a man-made tissue, such as a bioprinted tissue. “Artificial tissue” and “tissue construct” are used interchangeably herein. “Tissue progenitor” refers to an ensemble of cells that are capable of forming a tissue that can carry out a specific function, upon culturing, induction, or other manipulation steps. In some embodiments, a tissue progenitor is a man-made (i.e. “artificial”) tissue progenitor. In some embodiments, the cells in the tissue progenitor are not connected to each other. In some embodiments, the cells in the tissue progenitor are partially connected to each other.

### 3D Plant Bioprinting Compositions and Methods

Patterns of transition from undifferentiated stem cells to specialized cell-types underpin eukaryotic development. For example, in the Arabidopsis root, these patterns are highly regular and well-characterized and have led to the identification of cellular interactions and regulatory factors that influence the timing and positioning of these transitions. However, until recently the ability to quantitatively measure and individually perturb these cellular interactions has been limited. Recent advances in single cell sequencing allow for the establishment of models of higher resolution and predictive power. Yet, these models of gene expression networks need to be integrated with knowledge from morphogen gradients and cellular interactions that play crucial roles in setting the rules of organ development. To this end, it is important to be able to monitor and manipulate key mobile regulatory factors (e.g., the phytohormone auxin). Accordingly, embodiments of the present disclosure provide integrated compositions, systems, and computational methods to dissect the rules and mechanisms by which coordinating inputs, such as gene expression and auxin signaling, to provide the basis for plant regeneration.

As provided herein, embodiments of the present disclosure isolate the connection between inputs and responses and identify the rules and system parameters governing complex systems composed of multiple cell-types organized in three dimensions. These highly complex arrangements ensure the robustness of cell-to-cell communication, patterning, and ultimately cell, tissue, and organ functions. In accordance with these embodiments, a systems-level approach was used, including gene regulatory network inference and mathematical modeling, to generate and test hypotheses about the rules governing canonical cell identity patterning. Integration of single cell gene expression analysis with bioprinting technologies as well as biosensors and optogenetic actuators, inform and test model predictions. By arranging plant cells in predetermined, high-resolution architectures, using fluorescent reporters to quantify small molecule (morphogen gradients), and performing single cell gene expression profiles and network modeling, the embodiments provided herein determine the critical spatial and temporal robustness of patterning and interaction among cells, determine the rules by which morphogens gradients are established across cells to predict differentiation and identity, and identify mechanisms regulating stem cell maintenance and differentiation into specific tissues.

Embodiments of the systems, compositions, and methods described herein provide a basis for efficiently engineering plants with specialized developmental properties, such as for example, increased vascular capability, thus enabling higher plant tolerance to fluctuating environmental conditions. Developing plants to exhibit any other advantageous properties can also be achieved using the systems, compositions, and methods of the present disclosure. For example, stem cells divide and differentiate to form all cell and tissue types of multicellular organisms. Key to the organized and reproducible patterns of cellular differentiation and developing tissues are dynamic, yet robust, regulatory signaling mechanisms. Lack of understanding of the rules governing cell-to-cell communication as well as the design principles of regulatory networks controlling the establishment of precise patterns of gene expression and morphogen gradients is a bottleneck to generating predictable plant developmental outcomes. Consequently, identifying the factors and key mechanisms regulating plant development provides strategic opportunities for remodeling plants and improving crop yield and biomass production for food, fiber, and fuel.

Embodiments of the present disclosure facilitate the identification of characteristics involved in cell-to-cell communication, tissue patterning formation, and robustness that instruct cell identity, as well as stemness and differentiation during organ formation. In particular, embodiments of the present disclosure use three-dimensional (3D) bioprinting and single cell expression analysis to investigate the interactions among cells, the robustness of gene expression important for stem cell fate and identity, and the regulatory mechanisms controlling tissue patterning. Embodiments of the present disclosure include manipulating the spatial arrangement of cells, thus providing a molecular framework for understanding tissue patterning driven by diverse positional rules and mobile morphogens. Embodiments of the present disclosure also help determine the rules by which morphogen gradients are established across cells to predict cell identity and differentiation. Using biosensors for cellular and subcellular spatiotemporal quantification of auxin, 3D bioprinting, and live-imaging, embodiments of the present disclosure provide important information into how morphogen gradients influence cell-to-cell interactions. Embodiments of the present disclosure also help identify regulatory mechanisms governing stem cell identity and differentiation into specific tissues (e.g., plant callus). Through network analysis and mathematical modeling, key regulators controlling robustness of gene expression can be identified. Integrating this gene regulatory network information with machine learning techniques allow for the connecting of gene expression boundaries to morphogen gradients, which is key to the emergence of biological complexity.

As described further herein, embodiments of the present disclosure effectively isolate the connection between inputs and responses and identify the rules and system parameters governing the robustness of developmental processes underlying cell fate determination and identity. Genome-wide approaches and mathematical modeling of regulatory networks with 3D bioprinting technologies and machine learning approaches as well as biosensors and optogenetic tools are be used to monitor the function of biological circuits over time and at cellular resolution. Isolating and arranging, at high resolution, plant cells in predetermined architectures (e.g., generation of minimal stem cell niches) allow for the measurement, quantification, and systematic determination of the rules that drive tissue pattern formation.

In accordance with these embodiments, mathematical model predictions, using the acquired quantitative data from gene expression and biosensor analysis, are used to identify the mechanisms that exist for cells to interpret morphogens and the importance of features such as network motifs in regulating cell-to-cell communication, provide a comprehensive description of the system properties that accurately capture the behavior of the system, and validate the network and/or pinpoint other system-level control points.

The experimental and computational approaches described above facilitate the establishment of predictive molecular models for how plant development occurs. For example, stem cells are present in all multicellular organisms and are considered the building blocks for different cell-types and tissues. The Arabidopsis root offers spatially- and temporally-oriented lineages (from stem cells to their differentiated progeny), providing a robust system to identify the emergent properties underlying cell-type specification, identity, and tissue differentiation. Importantly, disruption of the stereotypical cellular arrangement of the root via physical, mechanical, or laser ablation can provide information about the underlying rules of cellular reprogramming and reestablishment of morphogen patterning. However, interrogating cellular reprogramming and gradient reestablishment has thus far been limited by the challenge of manipulating individual cells.

The Arabidopsis root tip contains a number of stem cell populations that divide to form two daughter cells. One of the daughter cells becomes the new stem cell, while the other daughter cell differentiates into specific tissues. The stem cells are confined in a region of the root tip called the stem cell niche (SCN), which contains a set of cells called the Quiescent Center (QC) that are surrounded by the stem cell initials. The three stem cells adjacent to the QC divide shootwards to form the lateral and proximal root tissues and include: the lateral root cap/epidermis initials (LRC/EPI), the cortex and endodermis initials (CEI), and the vascular initials (VASC). Distal to the QC are the columella stem cells (CSCs). QC cells are relatively mitotically inactive and are known to provide stem cell maintenance signals. Accordingly, division of cells within the SCN is coordinated through cell-to-cell signaling mechanisms that incorporate small molecules and/or transcription factors as signaling molecules. Moreover, when the root tip is excised, including the region of the QC and all the stem cell initials, many of the remaining proliferating cells within the root give rise to new stem cells, creating a new SCN. Thus, these cells form new cell-types distinct from their original cell fate, exemplifying the potential of plant cells to respond to local signals (e.g., signals within the local microenvironment such as auxin and mobile transcription factors). Embodiments of the present disclosure utilize the unique properties of Arabidopsis root stem cells to explore the signaling mechanisms driving cell fate determination. However, as would be recognized by one of ordinary skill in the art based on the present disclosure, any plant can be used according to embodiments of the compositions, methods, and systems provided herein.

Generally, single cell gene expression analysis and mathematical modeling of cell-to-cell regulatory networks can offer the advantage of identifying, within and across cell-types, the causal relationships of genes underlying the emergent behavior of the system. The spatiotemporal deposition and monitoring of cells via 3D bioprinting and biosensors, respectively, allow for high resolution and control over experimental variables, such as cell position, cell density, and morphogen gradients. Additionally, morphogen gradients are driven by local cell-to-cell interactions. Understanding how and why auxin is patterned is a challenge in plant biology. Using a direct biosensor in controlled 3D printed cellular arrangements, embodiments of the present disclosure provide a novel mode-of-action and make long-standing questions newly tractable. Thus, the precise placement of plant root cells in predetermined architectures (using 3D bioprinting), monitoring of molecule gradients (using biosensors), single cell gene expression analysis, and network modeling allow for the identification of the rules of cell-to-cell communication and tissue patterning required for organ formation.

The differential localization of morphogens and small molecules, such as auxin, generates gradients that regulate multiscale growth coordination. Dynamic changes in auxin levels translate into a wide range of transcriptional outputs to ensure robust stem cell activity, including stem cell fate and cell differentiation. Importantly, it is known that the auxin maximum at the tip of the root is essential for the positional organization of the stem cell niche. Several mathematical models have been developed to better understand the link between auxin patterning and plant development. Current models reproduce the auxin gradient and maximum that is found within the root tip by integrating the PIN-FORMED (PIN) proteins and auxin as upstream regulators. However, challenges remain for quantitative detection and spatiotemporal monitoring of small molecules. For example, auxin concentrations and responses are most often assessed directly at the organism or organ level and only indirectly at the cellular level. Descriptions of cellular auxin dynamics are mainly based on semi-quantitative and indirect measurements using the well-known auxin responsive promoter DR5 and DII-Venus reporter systems. While these reporters have provided valuable information regarding auxin patterning, they rely on nuclear auxin signaling and thus do not directly measure auxin. As a result, these reporters are unable to make predictions about extracellular auxin concentration or gradients of intracellular concentration and are more susceptible to artifacts stemming from changes in auxin signaling independent of auxin concentrations. In addition to limitations in auxin measurement, auxin perturbations are often genetic mutants or hormone treatments that are highly pleiotropic owing to the multifunctional nature of auxin programming. These limitations can be circumvented by mapping and then perturbing auxin patterns at high-resolution to interrogate how these patterns are determined and how they relate to stem cell activity and differentiation. More specifically, direct biosensors can increase the resolution of auxin measurement and optogenetic actuators are capable of introducing cell-specific perturbations. For the latter, genes encoding for light-sensitive proteins are introduced into specific cell-types or organs to precisely monitor activity using light signals that can be delivered with subcellular precision. However, the application of optogenetic systems has been limited in plants due to: 1) the need to grow plants in broad spectrum light and 2) the presence of many endogenous photoreceptors in plants that are highly active in reprogramming plant development. Development of sensors and optogenetic actuators capable of directly measuring and manipulating cellular morphogen concentrations allow for a complete exploration of the role of morphogen gradients in determining cell fate.

Embodiments of the present disclosure employ 3D bioprinting technologies that allow for the precise, high-throughput deposition of multiple cell-types, biomaterials, and growth factors simultaneously at a high resolution. These enabling technologies have been applied for tissue engineering, efficient screening, and personalized treatment in mammalian studies. The ability to 3D bioprint tissues and organs as well as other functional cellular/biomaterial structures for therapeutic, diagnostic, and research applications is already revolutionizing the medical field. For example, human induced pluripotent stem cells have been bioprinted to study cell fate, phenotypic variation, and tissue regeneration. Similarly, 3D bioprinting has the potential to revolutionize the plant biology field. While bioprinting either plant or animal tissues, it is imperative to recapitulate the attributes of native tissues at length scales ranging from sub-micron (e.g., interfaces of adjacent cells, biomaterials, etc.) to macro (e.g., tissue- and organ-specific 3D geometry). Just as with conventional 3D printing, there are multiple methodologies that can be used to perform 3D bioprinting and recapitulate specific geometrical arrangements, including inkjet, extrusion, laser-assisted, photopolymerization, and scaffold-free spheroid-based bioprinting. Specific arrangements are made possible based on methodology selection, such as continuous printing, drop-on-demand printing, and even single cell printing. For single cell printing, in which individual cells are positioned in three dimensions with micrometer resolution, laser-based printing uses laser pulses sent every nanosecond to deposit microscopic droplets of bio-ink with picolitre accuracy. By combining 3D bioprinting with novel computational modeling, embodiments of the present disclosure not only enable understanding of the key structural factors driving cell fate and system robustness but also allow for the prediction of new architectures to engineer plants with desired traits and features.

In some embodiments, emergent system-level characteristics involved in cell-to-cell communication, patterning formation, and robustness that instruct stemness, differentiation, and growth of an organism are used to enhance plant regeneration efficiencies of elite lines, and significantly reduce the time and cost-capability curves of the breeding market. For example, regenerative and recalcitrant germplasms of various plants can be used as a model crop for protocol optimization and future upscaling. Germplasm can be produced or obtained. Positional cues, such as cell density, cell number, and geometrical structure (e.g., precise spatiotemporal deposition of cells) strongly influence the regenerative capacities of plants. Using 3D bioprinting, these parameters are evaluated and optimized using germplasms from plants known to have efficient plant regeneration. These germplasms are also used to achieve fast regeneration rates. Manipulation of the spatio-temporal placement and deposition of cellular materials can strategically and programmatically guide biological outcomes.

Additionally, computational models are developed to systematically predict the most influential 3D bioprinting parameters that drive plant regeneration optimization, including fast and high-efficient regeneration. By harnessing collected data (e.g., gene expression data) parameters including cell density, type, and arrangement are quantified and explored. Experiments driven by model predictions are used to further improve the model itself. In some embodiments, an array of specific cells, which may undergo genome editing via the CRISPR/Cas9 system, can be used to produce a product of interest. With this approach, time-consuming genetic manipulation can be reduced.

Embodiments of the present disclosure can also be used with carrot plants. Carrot (Daucus carota) is a biennial crop, generating seeds every two years. Somatic embryogenesis of carrot is well-studied, making carrot an optimal model system for beginning development of 3D bioprinted seed genesis pipelines. To achieve accelerated seed genesis, protoplasts from different carrot tissues are generated and resuspended in a bio-ink supplemented with hormones. The bio-ink is 3D bioprinted on slides compatible with confocal imaging to track the development of the bioprinted embryo. The ability of each tissue (e.g., young leaves, mature leaves, roots, hypocotyl, and meristematic tissues) to regenerate in the presence of hormones can be evaluated. Additionally, to eliminate reliance on growth hormones during seed genesis, the intrinsic properties of the endosperm and its influence on embryonic development is exploited. Through manual dissection under a stereoscopic microscope, sufficient endosperm cells can be obtained to 3D bioprint around the somatic embryos and supplement further development. Printing layouts such as layering optimizes endosperm placement. Further, isolated carrot cells are 3D bioprinted in combination with the endosperm in a hormone-free, semisolid matrix. Several critical properties have been shown to be of importance for the development of a somatic embryo including sucrose concentration, calcium, nitrogen source, and pH. As the effect of these conditions on somatic embryogenesis is delineated, this information is used to generate optimal hormone-free conditions for seed genesis.

This strategy can be applied to efficiently propagate multiple species of crops and eliminate the time and monetary limitations of current practices. Crops such as triploid tomato, melon, sunflower, lettuce, and soybean can be selected to scale up the process any of the processed disclosed herein. Control of somatic embryogenesis via 3D bioprinting opens up nearly endless new possibilities in crop breeding: if single cells can be genetically transformed and induced to differentiate and reform whole plants via embryogenesis, then transformed plants can be quickly and easily obtained without the need for added hormones.

Embodiments of the present disclosure also include the ability to generate what might be considered hybrids by programmatically combining stem cells from different plants and to predict the desired traits based on the cells chosen from each species. For example, efforts have focused on improving the yield of perennial grain crops similar to, or even higher than, annual grain crops. The successful development of these perennials carries inherent benefits, such as mitigating soil erosion, reaching deep-soil water reserves with expanded root architecture systems, and reducing the environmental and economical impacts associated with yearly farming. Currently, the generation of such plants requires hybridization, genetic research and generations of successive plantings. Embodiments provided herein include 3D-printing techniques, which speed up the process of hybrid generation and redesigns plants that combine cells from different plant species with predictable and desirable traits. The application of plant 3D-bioprinting yields immediate and lasting agronomic and economic advantages, all without the stigma of genetic modification.

Human existence depends on food, fiber, shelter, and fuel from plants. In order to meet demands of a growing population, it is important effectively and sustainably increase crop production, which requires game changing advances in plant design. Regulation of plant growth and biomass is dependent on the continuous generation of stem cells that differentiate to form all the cells, tissues, and organs of the plant. Understanding the spatial and temporal control of stem cell self-renewal and their differentiation is critical for generating predictable outcomes and redesigning plants for future needs. 3D-bioprinting technology is particularly well suited for layer-by-layer, micro-structured geometries. Although it has not previously been applied to fabrication of plant tissues. 3D-bioprinting technology can precisely pattern the multi-layer stem cell niche thus optimize and control cell distribution and ultimately plant characteristics. The use of 3D-printing capabilities facilitates the testing and understanding of the critical three-dimensional spatial interactions of cells within the niche, and also allows for the redesign of novel plant systems based on digitally modelled communication networks.

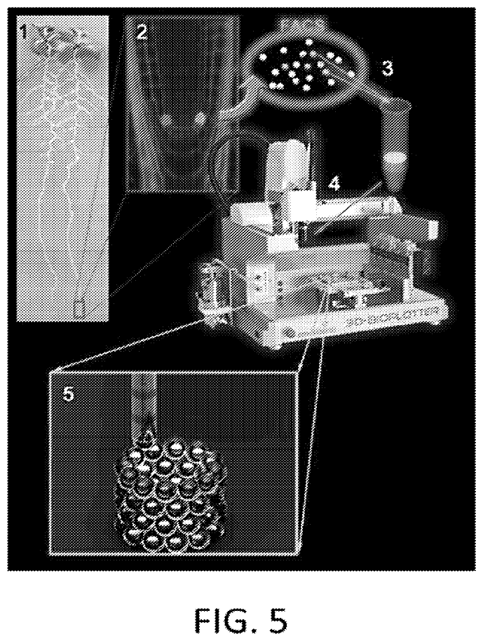

Plant cells can be easily isolated and manipulated because they do not move. Moreover, plant stem cells divide in a stereotypical manner and their tissues are organized into cell layers where entire cell lineages are spatially restricted. Despite this advantage. 3D-printing technologies have thus far been limited to animal tissues. Thus, embodiments of the present disclosure include the following: (i) isolation of the stem cells of the model plant Arabidopsis for which fluorescent markers for its spatially confined stem cells are available, and (ii) utilization of cutting edge 3D-bioprinting technology to precisely distribute these cells layer-by-layer to form a three dimensional functional structure (FIG. 5). The critical cellular arrangement for reconstructed stem cell niches can be determined to learn how these cells sustain their own growth and support the growth of their progeny. Since previous efforts have shown that complete plants can be regenerated from cultures of undifferentiated cells and tissues with exogenous treatment of appropriate growth cues, the resulting “artificial” stem cell-like 3D niche mimics various functional aspects of plant stem cells. The 3D-bioprinted systems provided herein demonstrate that plant regeneration and organogenesis from these stem cells are dependent on the microenvironment of the stem cell niche itself. The advantages of this, over classical plant cell culture, includes the ability to predictably redesign plants based on models of the spatial communication network, and potentially combine species without generating aneuploids with unpredictable and undesirable traits. The selection of specific materials (e.g., different stem cells) coupled with the precision of the 3D bioprinting systems allow for the generation of plants with predictable and desirable traits. 3D-printing of living cells allows for the generation of functional plant tissues and organs, and pave the way for custom designs in improvement of agronomically relevant traits.

To replicate many of the complex micro-scale physical and chemical features of the stem cell niche, and eventually of the whole plant, embodiments of the present discourse include gene expression analysis and in vivo imaging to systematically identify what physical, chemical, and biological features (e.g., cell position, distance, density, geometry, substrate, gene networks, etc.) vary with microscale resolution and how these features change with time. Experimentally determined parameters are essential for generating models that predict the correct spatial arrangement of cells, their geometry and density, and their physical and chemical micro-environment, which eventually ensures the functionality of the system.

Once the desired cell-types and tissues are formed, distinct plant systems are developed: from engineered plant cells for the generation of perennial grain crops and of environmentally improved crops, to engineered plant cell platforms for the production of plant-derived goods. Using the 3D-bioprinter, isolated plant stem cells are precisely deposited in a predetermined spatial location, which, through mathematical modeling, is predicted to improve a specific characteristic. Therefore, instead of genetically manipulating a plant, stem cells are printed layer-by-layer into a 3D structure to spatially control their tissue patterns. For example, if the root inner vascular layers are most responsive to iron deficient conditions, then additional stem cells layers are printed specific to the phloem and xylem cells to facilitate iron absorbance and mobilization through the plant.

Results obtained in accordance with the embodiments of the present disclosure reveal that the cell-to-cell interactions/orientations are important for the maintenance of stem cell niches and for the generation of redesigned plants with enriched functionality. In some embodiments, a basic understanding of factors regulating development and differentiation in the model system Arabidopsis, for example, provides strategic opportunities for remodeling plant development and improving crop yield and biomass production for food, fiber and fuel.

In accordance with the above, embodiments of the present disclosure include the use of 3D bioprinting devices and systems for plants. 3D bioprinting is a state-of-the-art technology that has been applied for tissue engineering, high-throughput screening, and personalized treatment in mammalian applications. For example, human induced pluripotent stem cells have been bioprinted to study cell fate, phenotypic variation, and tissue regeneration. By precisely depositing living cells and designing structural matrices, such as microfibrillated cellulose, whole tissues can be re-engineered. Plant science would benefit greatly from 3D bioprinting as a technological tool for fundamental research, translational research, and industrial applications. With 3D bioprinting the minimal cues, positional effects, and signaling pathways key for plant developmental processes such as cell division or differentiation could be studied. Identifying structural polymers, signaling molecules, or cell types that ensure efficient plant regeneration could have a large number of industrial applications. Thus, embodiments of the present disclosure include performing 3D bioprinting through the application of one or multiple methodologies for the precise spatial deposition of biologically active material. Available methodologies range from conventional (e.g., inkjet, extrusion, and laser-assisted) to newly emerging (e.g., ultrasound, photopolymerization, and scaffold-free spheroid-based). Depending on the selected methodology, arrangements such as continuous bioprinting, drop-on-demand bioprinting, and even single cell bioprinting can be performed.

Thus, in accordance with the above description, embodiments of the present disclosure include a matrix comprising a plurality of bioprinted plant cells. In accordance with these embodiments, the plurality of bioprinted plant cells are deposited in the matrix according to a pre-determined spatial pattern. In some embodiments, the matrix is three-dimensional (3D) or two-dimensional (2D). In some embodiments, the matrix comprises at least one of agar, hydrogel, nanofiber, plant biomaterials, and any combinations thereof.

In some embodiments, the plurality of plant cells includes at least one plant cell obtained from a meristematic region of a root and/or at least one plant stem cell. In some embodiments, the plurality of plant cells includes at least one stem cell obtained from a shoot and/or root stem cell niche. In some embodiments, the plurality of plant cells includes at least one cell obtained from a shoot and/or root apical meristem of a plant species.

In some embodiments, the plurality of plant cells are deposited in the matrix according to a pre-determined temporal pattern. In some embodiments, the matrix further includes at least one of a hormone(s), a phytohormone(s), a nutrient(s), an antibiotic(s), a prebiotic(s), a probiotic(s), a peptide(s), a polypeptide(s), a protein(s), a other growth factor(s), and any combinations thereof. In some embodiments, the pre-determined spatial pattern induces the plurality of plant cells to form a callus or a microcallus.

In some embodiments, at least one of the plurality of plant cells is naturally occurring. In some embodiments, at least one of the plurality of plant cells is genetically modified to include one or more desirable traits.

In some embodiments, the plurality of plant cells are deposited using a bioprinting device, as described further herein. In some embodiments, the plurality of plant cells are deposited in the matrix according to at least one additional pre-determined metric selected from cell number, cell density, cell-type, cell arrangement, and bioink composition (see Examples below).

Embodiments of the present disclosure also include a plant callus or microcallus formed from any of the plurality of bioprinted plant cells described herein. Embodiments of the present disclosure also include a plant organ, organoid, or tissue formed or derived from any of the bioprinted plant cells described herein.

Embodiments of the present disclosure also include a system for bioprinting plant cells. In accordance with these embodiments, the system includes a plurality of plant cells, a matrix whereupon the plurality of plant cells are deposited, and a bioprinting device.

In some embodiments, the bioprinting device deposits the plurality of plant cells in the matrix according to a set of pre-determined instructions. In some embodiments, the bioprinting device includes a processor component and a software component. In some embodiments, the software component includes the set of pre-determined instructions for bioprinting the plurality of plant cells. In some embodiments, the processor component executes the set of pre-determined instructions.

In some embodiments, the set of pre-determined instructions includes spatial and temporal information for depositing the plurality of plant cells in the matrix. In some embodiments, the spatial and temporal information is based on a computational model that includes gene expression data, cell number, cell-type, rate of cell division, and any combinations thereof.

Embodiments of the present disclosure also include a method of producing a plant callus. In accordance with these embodiments, the method includes bioprinting a plurality of plant cells in a matrix, and culturing the bioprinted plant cells in the matrix to induce the formation of a callus. Embodiments of the present disclosure also include a plant callus formed using these methods, as well as a plant organ, organoid, or tissue, as described further herein.

### EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

Stem cell fate determination depends on positional information from local cell-to-cell interactions and mobile signals, such as small morphogens or mobile proteins. However, deciphering the requirements for the establishment and maintenance of positional information has been limited by the challenge of manipulating individual cells. Thus, embodiments of the present disclosure utilize 3D cell-bioprinting techniques to deposit cells in specific cellular architectures (e.g., accurately deposit cells into precise geometries with the goal of creating anatomically correct and/or rearranged root SCN structures). Embodiments of the present disclosure include methods to print plant cells in semi-solid media, such as a matrix. Protocols have been developed to monitor cells for survival and functionality, including visualization of cell wall formation, cell identity, and cell division. To evaluate and analyze printed cells, fluorescein diacetate (FDA) and calcofluor white are used, which stain for viability and cell wall formation, respectively.

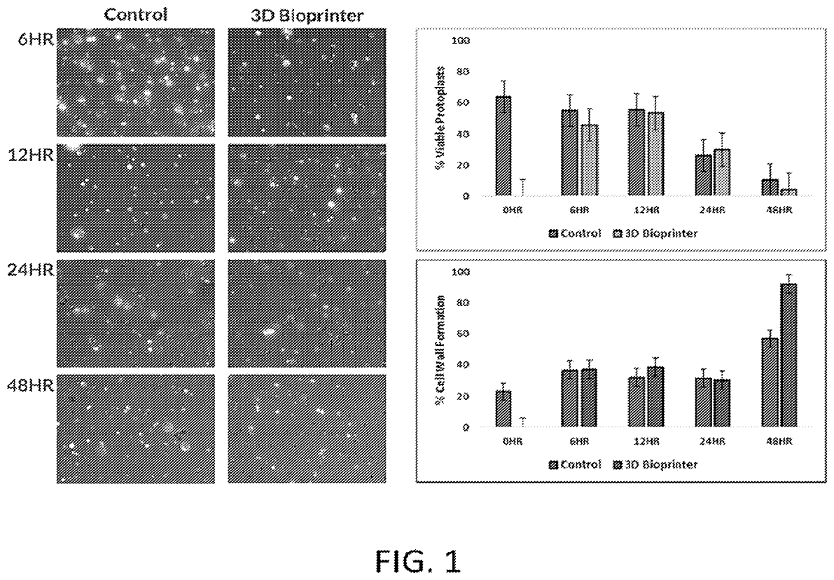

Experiments conducted indicate that approximately half of the cells that undergo protoplasting and printing are viable (FIG. 1). After 24 hours, the number of viable cells (>35%) still allows for further analysis, including monitoring for cell identity markers and cell division (FIG. 1). Additionally, results demonstrate that these viable cells retain their original functionality, including the expression of markers for cell identity, such as pSCR-SCRmCherry (FIG. 1), which is specifically expressed in CEI and endodermal cells and continues to be expressed for at least 48 hours after bioprinting. These results demonstrate that cells that have been bioprinted maintain their cellular identity and function.

Additionally, image analysis systems have been developed using Matlab that automatically detects cells using confocal imaging (FIG. 1). A computer vision system, initially developed for lightsheet microscopy, extracts, analyzes, and compares high-dimensional dynamic spatial and temporal cellular data, (e.g., 3D bioprinted cell architectures across multiple time points). As such, while performing cell segmentation of 3D images, data on viable cells are captured, whereas other debris and particulates are rejected. This facilitates automatica monitoring of cell growth and division, thus enabling efficient and accurate cell identification in 3D bright-field and fluorescence images across multiple time points.

To identify positional information, cell-to-cell interactions, and mobile signals that regulate cell fate and identity, embodiments of the present disclosure apply 3D bioprinting, auxin concentration manipulation, and single cell expression profiling. Within this system, stem cells are isolated and 3D bioprinting technology is utilized to deposit cells in cellular architectures that reflect the Arabidopsis SCN. Additionally, the systems provided herein facilitate the characterization of the influence of auxin on the development of 3D bioprinted cellular arrangements to define the rules underlying cell identity that ultimately provide the basis for plant regeneration.

Cell-lineage-specific markers and fluorescence activated cell sorting (FACS) are used to isolate different stem cell-types, along with various other markers, such as those specific for cell identity and differentiation. These include, but are not limited to, pWOX5:GFP, pCYCD6:GFP, pFEZ:FEZ-GFP, pTMO5:3xGFP, pCVP2:NLS-VENUS, PAGL42:GFP, pSCR::SCR-mCherry, pEPM::dBOX-YFP, pC2::YFP, pNEN4::hYFP and pHCA2::erRFP, marking QC cells, CEIs, LRC/EPI, xylem, CSCs, protophloem, SCN, phloem initials, sieve element cells, and cambium. Various other markers can be used, as would be recognized by one of ordinary skill in the art based on the present disclosure. The expression profiles of these marker lines have shown that transcriptional profiles are specific to stem cell-type lineages.

After FACS, these plant protoplasts are 3D bioprinted in spatial organizations designed to mimic the arrangement of cells within the Arabidopsis root SCN, providing a platform for experimentally querying its fundamental properties. For example, CEIs and vascular initials, and CSC cells are positioned proximal and distal to QC cells, respectively, via the cell-bioprinting process. These bioprinted SCNs resemble the spatial arrangement of the SCN in planta, thus providing a control system, which is useful when generating plants.

After printing, live-imaging of the 3D bioprinted SCNs is performed every 24 hours for at least 9 days, allowing for the monitoring of cellular division. Data provided herein demonstrates that Arabidopsis stem cells divide every 18-24 hours: cellular divisions of bioprinted cells take place within the same time frame. Images acquired over multiple time points are analyzed with imaging systems described herein to automatically monitor cell development and division.

Through Corrected Total Cell Fluorescence (CTCF) measurements, the intensity of cells expressing the fluorescent markers are quantified. Conserved and stereotypical spatial order of cells represents the stable state of the system. Generally, this state is realized by cellular interactions and is robust with respect to perturbations. To test this and to determine the key aspects of cell-to-cell interactions, complex 3D cellular relationships are determined using bioprinting, as described herein. The 3D arrangement of cells can be organized as an SCN by perturbing their spatial positioning. Repositioning of cells allows for the perturbation of cellular interactions, thus allowing molecular data to be acquired, which helps determine the startup or shutdown of interactions governing, for example, stem cell identity. For example, whether a vascular cell initially changes identity when placed at the spatial position of a columella cell can be discerned. CSCs can be placed proximal to the QC cells and vascular initials distal to the QC. Cell-bioprinting is followed by single cell gene expression (scRNAseq) analysis to determine changes in gene expression. Whereas gene expression analysis of a bulk population of cells can obscure specific trends by averaging the gene expression of multiple cells together, single cell analysis captures the high variability seen on a cell-to-cell basis. Collection of single cells from the 3D bioprinted SCNs for scRNAseq is performed using developed and commercial systems, such as capillaries and/or punching probes. The Monocle 3 tool can be used analyze the obtained scRNAseq data, as well as previously developed bioinformatic platforms. These methods include clustering cells based on gene expression, tracking their trajectories over time, and identifying differentially expressed genes (DEGs) in different cell-types and over time. Tracking expression across cells captured at the same time but from diverse spatial arrangements facilitates an understanding of how perturbations in positional information affect cell-to-cell interactions.

Bio-functional 3D bioprinting ink materials, matrices, and related compositions (e.g., semi-solid agar and/or gel scaffold and support materials) of the present disclosure allows for the cells to be exposed to instructive signals to induce a desired cellular response. As these efforts are often informed by the native biomolecular signals which instruct cell-to-cell communication in controlled spatial and temporal manners, bio-inks and related compositions are designed with a range of auxin concentrations and gradients. Moreover, adding and/or depleting auxin concentrations enables a better understanding the role of auxin in regulating the early signals instructing robustness and cell fate transition. In accordance with these embodiments, cell fate determination is likely be regulated by the concentration of growth factors that can be locally influenced by both intra- and intercellular dynamics. To test this, auxin content can be modified directly in the support materials and matrices and auxin mobilization across cells. For auxin mobilization, carbodiimide crosslinking and benzophenone photoimmobilization chemistries are sequentially employed to immobilize intracellular auxin, thus interfering with intra- and intercellular auxin-driven communication. Further, the singular and combined effects of supplementing and/or depleting cells from auxin are investigated using a preprinting composition containing different concentrations of auxin, photoimmobilization chemistry strategies, and by monitoring the activity and expression of lineage-specific markers. As such, because auxin acts upstream of the CYCD6:1 and WOX5 transcriptional networks, pWOX5:GFP and pCYCD6:GFP expression is monitored and quantified, and the time points at which auxin concentrations and related gene expression profiles intersect can be determined, which instructs stem cell fate determination.

Because auxin can elicit diverse cellular responses depending on the cell-type and local context (e.g., cell position), functionalized bioprinting support materials are used both in the “native” 3D arrangement of the SCN as well as in rearranged SCNs. To monitor the cell identity of printed vascular initials through cell-type specific markers, such as pEPM: dBOX-YFP, pC2::YFP, pNEN4::hYFP, and pHCA2::erRFP, CTCF measurement over time is used, as described herein. Additionally, although auxin signaling has been placed downstream of the SHR transcriptional network important for cell-type identity, cell-type specific responses to auxin have been identified. To understand the relationship between auxin and cell identity, PLT transcript accumulation and the expression of SCR and SHR, which are known regulators of stem cell identity are monitored. The overlap of PLT, SHR, and SCR expression domains provide positional information for the root SCN and acquiring gene expression data from pPLT2::PLT-YFP, pSHR::SHR-GFP, and pSCR::SCRmCherry via scRNA-seq and CTCF measurements facilitate an understanding of whether auxin also plays a role in regulating cell identity. Modification of auxin concentrations and quantification of related gene expression provided the ability to investigate the contribution of auxin signaling to the rules governing cell fate and identity. Accordingly, the methods and systems described herein can also be applied to other plant growth factors, hormones, and cellular modulators, in addition or as an alternative to auxin.

Based on the data obtained thus far relating to plant cell-bioprinting, it has been determined that 3D bioprinting processes and systems allow for the deposition of cells in pre-determined spatial organizations to facilitate plant regeneration. Additionally, fabrication of scaffolds and matrices can be customized to each cell-type to allow for precise arrangements of cells and to provide structural support. Diverse scaffolds and matrices can be used, and can include but are not limited to, semi-solid media using different bio-ink compositions, scaffolds with cellulose and lignin, and growth media supplements. Additionally, 3D bioprinted structures and matrices need to allow for adequate transportation of nutrients and oxygen to the cells, the thickness of the 3D bioprinted organization is one important factor (e.g., <150 μm), in addition to the presence of channels/empty spaces throughout the scaffold or matrix. In some cases, a suboptimal cell survival rate after protoplasting and 3D bioprinting may require the addition of proliferation-stimulating proteins (e.g., phytosulfokine) to the growth media compositions. The isolated protoplasts can also be treated with a pro-survival composition that includes phytosulfokine (a peptidyl plant growth factor), auxins (2,4-D), cytokinins (thidiazuron), gibberellins, and folic acid (a vitamin to promote cell survival and division), as well as any other plant growth factors, hormones, and cellular modulators.

### Example 2

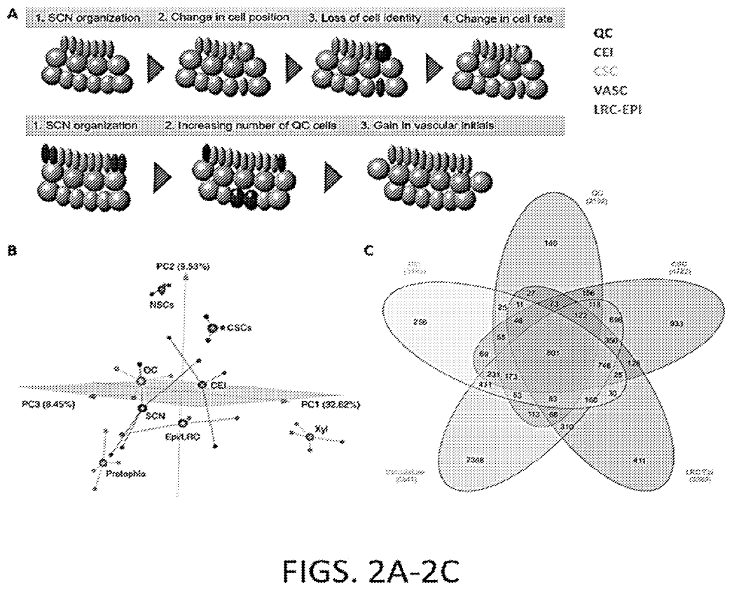

Beyond identifying DEGs based on gene expression data, predicting gene interactions in the form of gene regulatory networks (GRNs) enables an in-depth approach to determining key genes and making system-level conclusions based on network-wide trends. Computational systems have been developed and validated for inferring GRNs: GENIST, a Bayesian inference approach, and RTP-STAR (Regression Tree Pipeline for Spatial And Temporal Replicate data), a machine learning decision tree approach. Additionally, the framework of a mechanistic computational model that predicts cell identity based on parameters, such as starting position and gene expression, has been developed (FIG. 2A). Currently, the model incorporates Arabidopsis root stem cell expression data, spatial arrangements, and distance from the QC cells. The specific identities of cells within SCN have been individually resolved via principal component analysis (PCA) and gene enrichment analysis of stem cell transcriptional profiles (FIG. 2B) while retaining information on common genes and patterns (FIG. 2C). Using these models as a basis, a predictive model can be developed to explore and identify the robust developmental modules related to gene regulatory interactions that instruct cell fate determination and cell identity.

To identify genes, networks, and regulatory rules that play a key role in determining, for example, cell identity, publicly available high-resolution single cell expression data of the Arabidopsis root cells are used. Currently, scRNAseq data from the Arabidopsis root cell-types cannot discriminate between the different stem cell initials. However, with the scRNAseq data described herein, an analysis of the expression profiles of individual CEIs, vascular initials, CSCs, and QC can be performed separately. By analyzing the expression profiles of these individual stem cells, key transcription factors (TFs) that regulate the patterning from, for example, QC to CEI, can be identified. Bayesian inference and machine learning decision tree computational systems can be used to predict GRNs using these key TFs and their expression profiles. The available single cell datasets can be reanalyzed as follows: 1) cluster populations of cells that have similar expression profiles with PCA: 2) attribute cell identities to each cluster by comparing cell-type specific marker genes across the identified cell populations: and 3) identify trajectories of the cell populations (e.g., lineage relationships between cell populations).

Additionally, using modified Shannon entropy (MSE), an unsupervised approach for selection of DEGs, genes enriched in the different cell populations identified by the PCA are selected. The results of this analysis provide a comprehensive list of DEGs, including transcription factors (TFs), either unique to or shared between single cell populations, which act together in regulatory circuits to control complex signaling cascades underlying cell identity. Because of this, efforts can be centered on predicting causal relationships between TFs that show a stem cell-type specific expression pattern. Specifically, established computational approaches used to infer GRNs from RNAseq data can be used to process single cell gene expression profiles. Computational platforms and systems can iteratively generate decision trees of gene interactions based on gene expression data. This comprehensive network analysis results in cell-type specific GRNs.

Additionally, to determine regulatory links associated with specific behaviors of systems, network motifs (e.g., small subgraphs within the network) are analyzed. For this, the resulting GRNs are investigated with motif score analysis and outdegree calculations to identify enriched network motifs and key TFs, respectively. To understand the temporal dynamics of key TFs, a set of ordinary differential equations (ODEs) can be developed, which predict gene expression levels over time using gene expression data and predicted network interactions. Specifically, second-order ODEs or Hill-type kinetic equations can be generated, where unknown interaction kinetics are replaced by Hill parameters. Since many of the parameters (e.g., protein synthesis and degradation rates) cannot be directly measured experimentally, parameter estimation can be applied to approximate their values. Considering the probabilistic approach to parameter estimation takes into account the aspect of randomness in measured data to give a plausible range for desired parameters, a sensitivity analysis on this ODE model can be used to determine the robustness of the model, measure the uncertainty of its output in relation to changes in input, and identify potentially correlated model inputs. Overall, this qualitatively improves model(s) with regard to measurement errors and gaps in mechanistic knowledge. To validate the dynamic expression profiles predicted by the ODE model, the expression of these TFs are quantified over multiple time points in wild-type plants and compared with model predictions.

To confirm the regulatory role of the selected TFs identified with network analysis, loss-of-function lines (e.g., T-DNA lines from the Arabidopsis Biological Resource Center (ABRC)) are phenotypically analyzed (up to 30 TFs). The loss-of-function mutants that show a cellular phenotype (e.g., disorganization of the SCN) are further characterized, such as using gain-of-function lines from the TRANSPLANTA collection and, if not yet available, generating transcriptional and translational fusions for conventional functional analysis. In particular, to test and validate the ODE models, the expression of genes is quantified over time in T-DNA insertion lines and gain-of-function using RNAseq. Together, these characterization studies facilitate an understanding of how key TFs and the regulatory circuits they are involved in impact emergent behaviors in stem cells, such as stem cell identity.

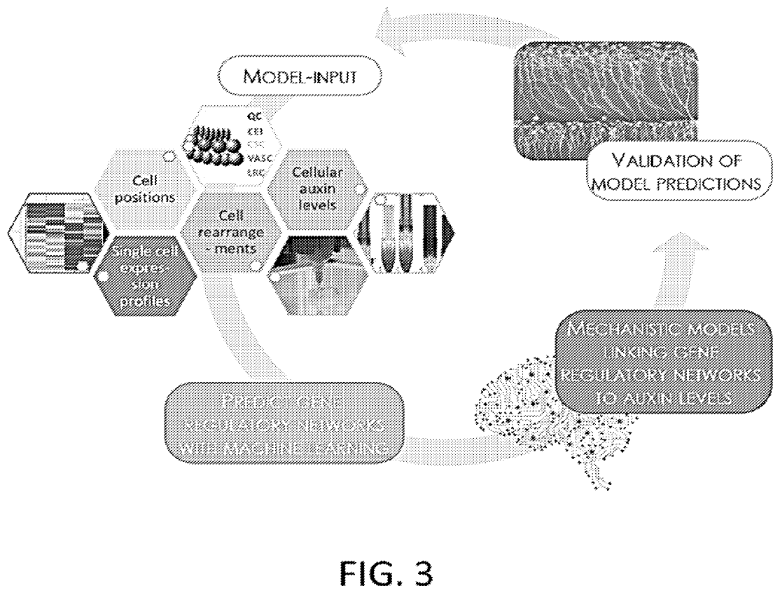

Advances in scRNAseq have led to the generation of models of higher resolution and predictive power. However, mobile regulatory factors and cellular interactions play crucial roles in the coordination of cellular responses and are not accounted for in current models. Fundamental understanding of the rules and mechanisms by which GRNs give rise to emergent system-level characteristics can be achieved by integrating knowledge from cell position, intra- and intercellular regulatory interactions, and local environment signals (e.g., auxin). To provide a system-level view of the gene regulatory interactions that instruct fate determination and identity, a computational model can be developed integrating data provided herein, specifically, data relating to: i) single cell gene expression profiles from scRNAseq: ii) auxin measurements at cellular resolution from the AuxSen biosensors: and iii) cell-type specific gene regulatory networks from Bayesian inference and machine learning decision trees can be used as input data to the model(s). Due to the multiscale, heterogeneous nature of the available data (e.g., multiple sources at different temporal and spatial scales), machine learning models can be used (FIG. 3).

Accordingly, to predict cell fate given gene expression, regulatory networks, cell position, and auxin levels, support vector machine (SVM), random forest (RF), and regression analysis methodologies can be applied. The model(s) developed facilitate framing of the problem (e.g., with classification and regression) and allow for enhanced predictive power. Among the regression methods, the least absolute shrinkage and selection operator (LASSO) approach allows for the accommodation of the large amounts of multiscale data acquired, allow for the testing of many potential predictors/parameters of different signaling networks, and facilitate the identification of the most important ones in terms of model variability. Whether the integrated information coming from different sources improves the performance of the original network predictions that resulted from using only one data type (e.g., scRNAseq data modeled using Bayesian inference and machine learning decision trees) can be examined. To this end, Receiver Operating Characteristic (ROC) curves are used as performance measures in addition to SVM classifiers. Within this procedure, performance of the classifiers at each layer is tested by using similarity (kernel) matrices to weigh information according to what is considered most relevant for modeling the robustness of regulatory networks.

By incorporating gene expression data, gene regulatory interactions, relative cell positions within either native or perturbed cell arrangements, and cellular auxin levels into the computational framework, the identification of genes and regulatory networks that correlate with auxin levels and cell position can be determined, and thus be the basis for establishing stem cell behaviors, such as cell fate.

### Example 3

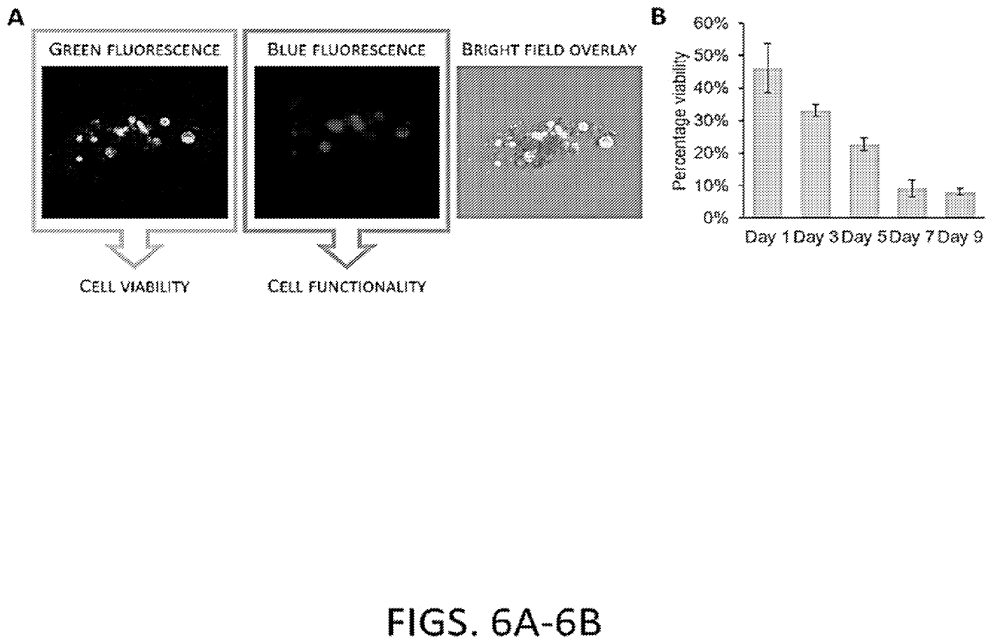

In one exemplary embodiment of the present disclosure, 3D bioprinting methods were developed using plant cells as bioink deposited in semi-solid media. Protocols were developed to monitor cells for survival and functionality, including visualization of cell wall formation, cell identity, and cell division. To evaluate and analyze printed cells, one protocol used fluorescein diacetate (FDA) and calcofluor white, which stain for viability and cell wall formation, respectively. Protoplasts were generated from different cell types, they were 3D bioprinted, and their survival, functionality, and cell identity were evaluated. More specifically, protoplasts were generated from Arabidopsis root meristematic and differentiated cells and brought to certain densities. As protoplasts are highly sensitive to environmental properties (e.g., pH and osmolarity) and shearing stress, a specialized bioink was developed, which contained a cocktail of growth hormones, sugar, mannitol to ensure a proper osmolarity, and 0.6% low melting agar to provide support for cell growth. However, the designed bioink could sustain cell viability up to 9 days (FIG. 6). When pretreating the protoplasts with a pro-division cocktail, cell viability increased, demonstrating that with the right environmental cues plant cells can be sustained over long periods of time. Moreover, after a certain number of days, some plant cells were dividing and generated microcalli. When 3D bioprinting cell lineage markers, it was found that a significant percent retained their cell identity (FIG. 6).

### Example 4

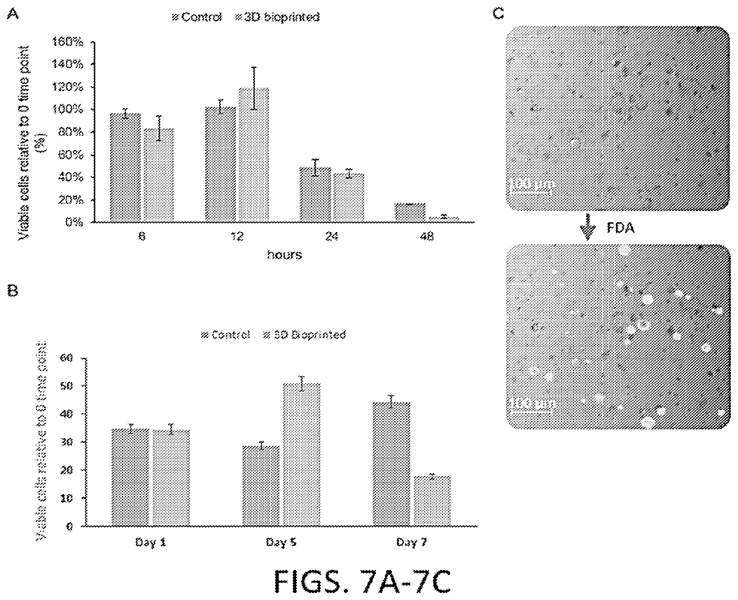

As shown in FIGS. 7-12, experiments were conducted to assess the viability, growth, and development of 3D bioprinted plants in accordance with the various embodiments of the present disclosure. In particular, experiments were performed to assess cell viability of 3D-bioprinted protoplasts for both Arabidopsis and tobacco plants. FIG. 7 demonstrates that cell viability of manual pipetted and 3D bioprinted protoplasts isolated from meristematic root cells is comparable. The protoplasts were bioprinted in PIM with 0.6% low melting agar. n=2-6 images with 50-200 cells per image.

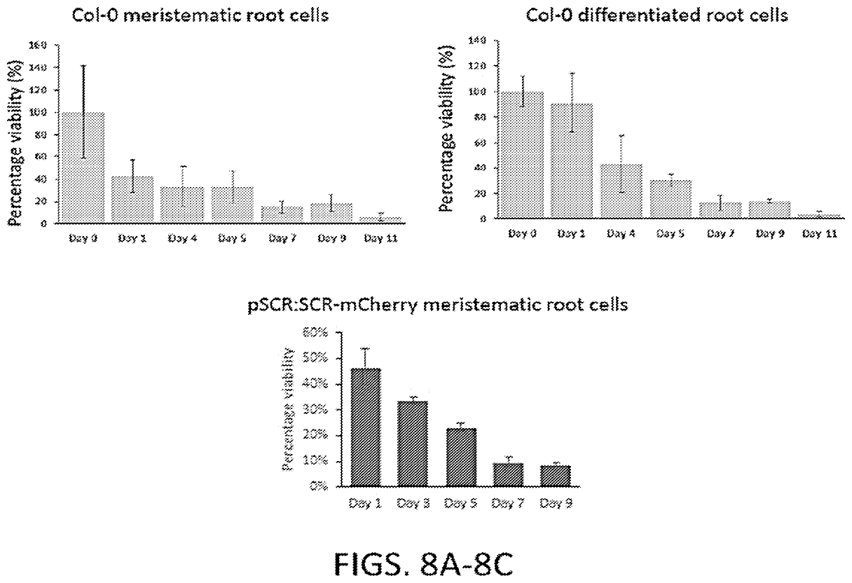

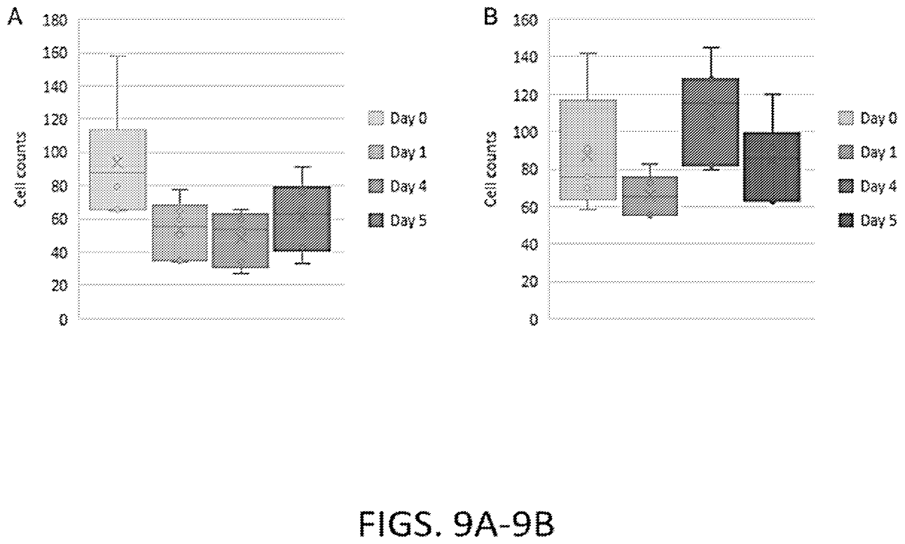

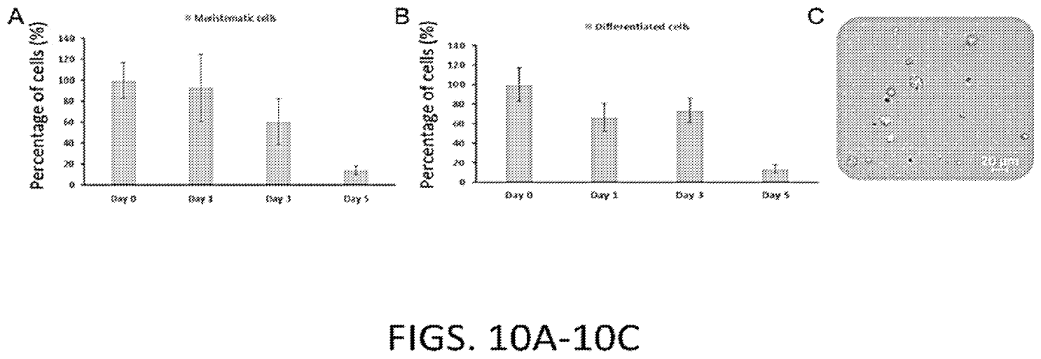

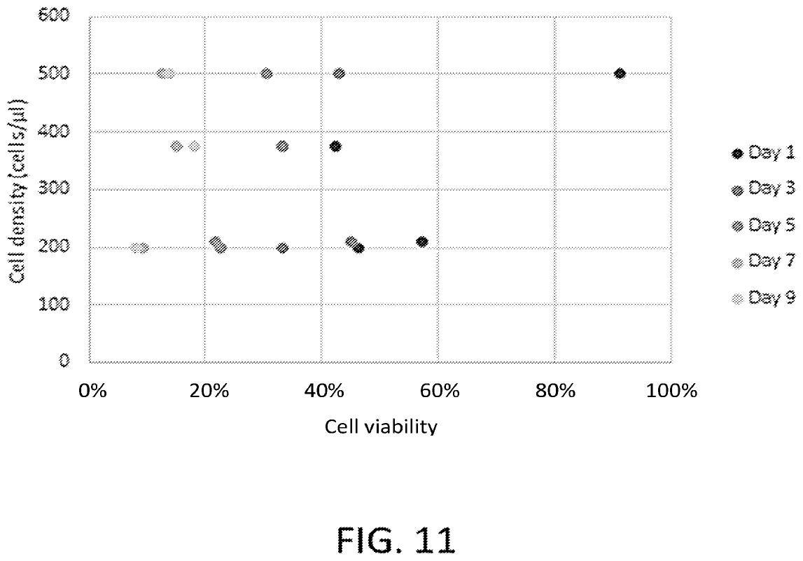

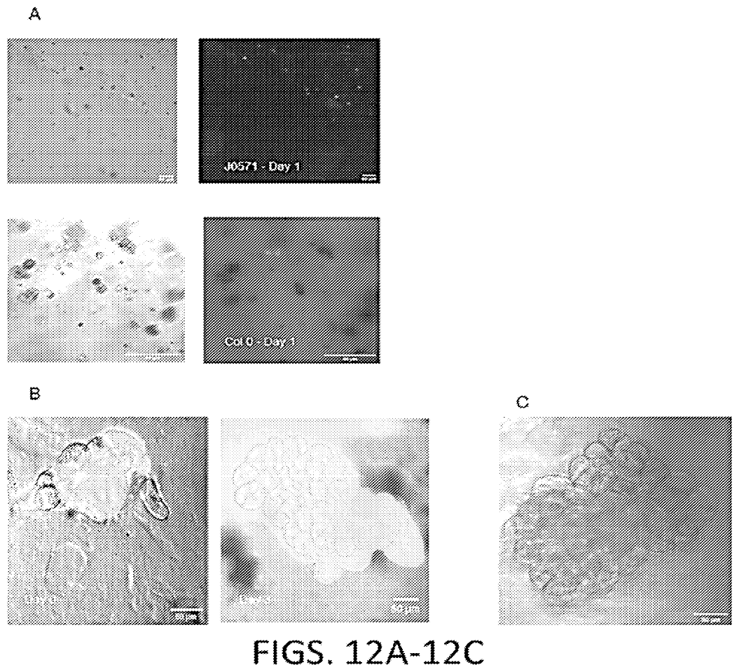

Additionally, as shown in FIGS. 8 and 9, cell viability (FIG. 8) and cell identity (FIG. 9) of 3D-bioprinted protoplasts was assessed over time using imaging analysis (from 0-11 days after bioprinting and imaged using the confocal microscope Zeiss 880). Cell viability was evaluated with fluorescein diacetate (FDA) staining. The protoplasts were bioprinted in PIM with 0.6% low melting agar. These data demonstrate an increase in the number of protoplasts, which points towards the generation of additional cells through cell division. Moreover, FIGS. 10 and 11 demonstrate that cell identity is retained for at least 3 days after bioprinting, and that cell viability is not necessarily correlated with cell density. Representative imaging data in FIG. 12 also demonstrate that the 3D bioprinting compositions and methods of the present disclosure produced successful encapsulation of multiple cell types in hydrogel matrices for Arabidopsis (FIG. 12).

As would be recognized by one of ordinary skill in the art based on the present disclosure, various system parameters can be used and modified as part of the 3D bioprinting methods described herein. For example, Table 1 below provides several adjustable parameters that can be used as a basis for performing the 3D bioprinting methods of the present disclosure.

TABLE 1
Exemplary 3D bioprinting adjustable parameters.

| | Representative | |
|---|---|---|
| Parameter | Experiment | Importance |
| Initial concentration | 4.26 × 10⁵ | High - high density of cells is |
| of cells | cells/ml | optimal for bioprinting |
| Final concentration | 0.6% | Moderate - allows cells to |
| of Agar | | divide and provides support |
| Size of bioprinting | 20-30 G | Moderate - smaller needle |
| needle | (~800 μm-150 μm) | dimensions/geometry will |
| | | damage cells |
| Pressure applied | 25 kPa | Moderate - higher pressures |
| to cells | | will shear cells |
| Number of droplets | 4 | Low - easily modifiable and |
| per well | | can be adjusted |
| Refreshment | Liquid bioink | High - prevents cells from |
| | | drying out, provides nutrients, |
| | | maintains osmolarity |

### Example 5

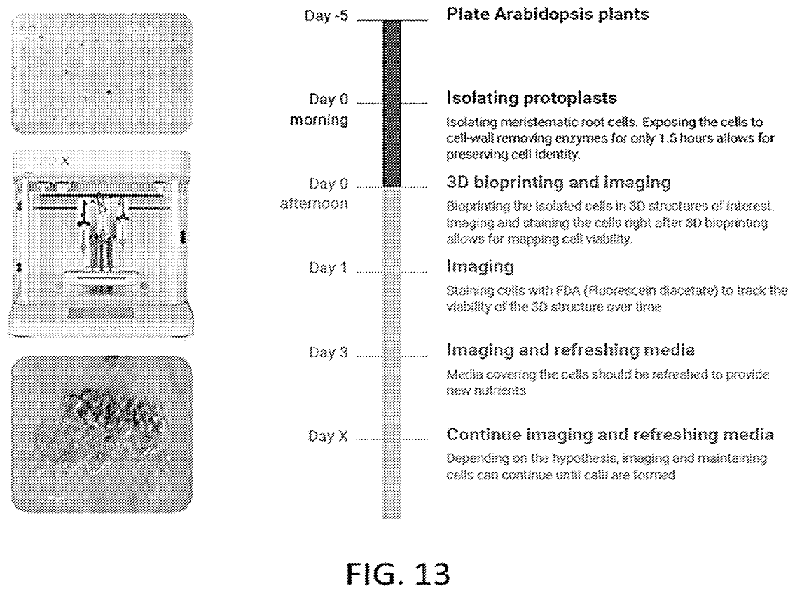

As shown in FIG. 13, the methods of the present disclosure can be adapted to 3D bioprint any plant cell/protoplast. For example, representative schematics of workflows for generating 3D bioprinted plant microcalli have been established for Arabidopsis, which can be subsequently modified for other plants. The protocol generally includes the steps of isolating protoplasts from the plant-of-interest, performing 3D bioprinting and imaging (e.g., determining viability), and applying fresh growth media and additional imaging until the desired growth stage is obtained.

In accordance with these methods, embodiments of the present disclosure also includes determining a bioink composition that is adapted to 3D bioprint protoplasts from a specific plant. Exemplary bioink compositions generally include, but are not limited to, cell culture media, growth factors, auxin, cytokinin, sucrose, and salts. In some embodiments, the bioink compositions include B5 media (see also below), which generally includes sucrose, 2.4-D, BAP, MES, CaCl₂-2H₂O, NaFe-EDTA, sodium succinate, folic acid, and phytosulfokine.

In some cases, bioink compositions can be developed to have components and properties that are particularly suited to a given plant. For example, bioink compositions for various plants can include, but are not limited, to the following:
(i) Mannitol: 13%, KH₂PO₄: 27.2 mg/l, KNO₃: 100 mg/l, CaCl₂): 150 mg/l, MgSO₄: 250 mg/l, FeSO₄: 2.5 mg/l, KI: 0.16 mg/l, 2,4-D: 0.1 mg/l, 6-BAP: 0.2 mg/l, NAA: 1 mg/l.
(ii) 0.4M Mannitol: 2% sucrose: ½ B5 medium: NAA: 1 mg/l: 6-BAP: 0.1 mg/l.
(iii) PIM: ½ B5 medium: 1.58 g, Sucrose: 2%, NAA: 1 mg/l, 6-BAP: 0.1 mg/l, Folic acid: 0.2 mg, Phytosulfokine: 10 nM.
(iv) MS-PPI MS-basal, 0.4 M glucose, 1 mg/l 2,4-D, 0.15 mg/l kinetin, pH 5.8: MSPPII MS basal, 0.2 M glucose, 0.05 mg/l 2,4-D, 2.0 mg/l NAA, 0.15 mg/l kinetin, 0.15 mg/l BAP, pH 5.8.
(v) MgSO₄-7H₂O, 746 mg/l, CaCl₂-2H₂O, 450 mg/l, L-glutamine, 50 mg/l, casein hydrolysate, 100 mg/l, coconut water, 20 ml/l, Dicamba, 3 mg/l, naphthylacetic acid, 0.5 mg/l.
(vi) PIM: ½ B5 medium: 1.58 g, Sucrose: 103 g, 2,4-D: 0.5 mg, MES: 0.1 g, CaCl₂-2H₂O: 375 mg, NaFe-EDTA: 18.35 mg, Sodium succinate: 270 mg.
(vii) PIM: ½ B5 medium: 1.58 g, Sucrose: 103 g, Dicamba: 3 mg/l, BAP: 0.3 mg, MES: 0.1 g, CaCl₂-2H₂O: 375 mg, NaFe-EDTA: 18.35 mg, Sodium succinate: 270 mg, Folic acid: 0.2 mg, Phytosulfokine: 10 nM.
(viii) MSR1: ½ MS medium 2.0 mg/l IAA, 0.5 mg/l 2,4-D, 0.5 mg/l IPAR 0.4M glucose.
(ix) liquid callus medium (½ MS medium supplemented with 0.4M mannitol, 30 g/L sucrose, 1 mg/L 1-naphthaleneacetic acid (NAA) and 0.3 mg/L kinetin).
(x) CPP medium (see, e.g., Dirks et al.), macro- and micro-elements and organic acids (see, e.g., Kao and Mychayluk et al.), vitamins according to B5 medium (see, e.g., Gamborg et al.), 74 g l−1 glucose, 250 mg l−1, casein enzymatic hydrolysate (Sigma), 0.1 mg l−1 2,4-dichlorophenoxyacetic acid (2,4-D), and 0.2 mg l−1 zeatin (pH 5.6, filter-sterilized).
(xi) PIM: Fe Citrate NH₄, AlCl₃, NiCl₂-6H₂O, Inositol, Panthotenate Ca, Biotin, Niacin, Pyridoxin, Thiamin, Folic Acid, Glucose, Mannitol, ‘2,4-D’, Thidiazuron (TZ), MES, Bromocresol purple (BCP).
(xii) MS media, 0.4 M mannitol, 20 mM MES, 20 mM KCl, 10 mM CaCl₂), 3.6 uM 2,4-D: 1.8 uM 6-BAP.
(xii) MS media, 1% sucrose, 0.6M sorbitol, 3.6 uM 2,4-D; 1.8 uM 6-BAP.
(xiii) PIM: ½ MS medium: 1.58 g, Sucrose: 103 g, 2,4-D: 0.2 mg, BAP: 0.3 mg, MES: 0.1 g, CaCl₂-2H₂O: 375 mg, NaFe-EDTA: 18.35 mg, Sodium succinate: 270 mg, Folic acid: 0.2 mg, Phytosulfokine: 10 nM.
(xiv) PIM: ½ MS medium: 1.58 g, Sucrose: 103 g, 2,4-D: 0.5 mg, MES: 0.1 g, CaCl₂-2H₂O: 375 mg, NaFe-EDTA: 18.35 mg, Sodium succinate: 270 mg.
(xv) MgSO₄-7H₂O, 746 mg/l, CaCl₂-2H₂O, 450 mg/l, L-glutamine, 50 mg/l, casein hydrolysate, 100 mg/l, coconut water, 20 ml/l, Dicamba, 3 mg/l, naphthylacetic acid, 0.5 mg/l.
(xvi) MSR1: ½ MS medium 2.0 mg/l IAA, 0.5 mg/l 2,4-D, 0.5 mg/l IPAR 0.4M glucose.
(xvii) MS-PPI MS-basal, 0.4 M glucose, 1 mg/l 2,4-D, 0.15 mg/l kinetin, pH 5.8: MSPPII MS basal, 0.2 M glucose, 0.05 mg/l 2,4-D, 2.0 mg/l NAA, 0.15 mg/l kinetin, 0.15 mg/l BAP, pH 5.8.
(xviii) MSR1: ½ MS medium 2.0 mg/l IAA, 0.5 mg/l 2,4-D, 0.5 mg/l IPAR 0.4M glucose.

### Materials and Methods

Plant lines and growth conditions. Arabidopsis thaliana seeds were sown and grown in a vertical position at 22° ° C. in long-day conditions (16-h light/8-h dark cycle) on 1× Murashige and Skoog (MS) medium supplemented with sucrose (1% sucrose total). For each experiment 250-500 mg of seed were wet sterilized using 50% bleach, 10% Tween and water, stratified at 4° C. for 2 days, and plated on 1× MS agar on top of Nitex mesh. The following lines were used in certain experiments of the present disclosure: Col-0, pSCR::SCR-mCherry.

Generation of protoplasts. To avoid contamination, all steps were performed in a laminar flow hood. Solution A was prepared as followed: 5.465 g of mannitol, 0.05 g of 0.01% BSA, 500 μL 0.2 M Magnesium chloride, 500 μL 0.2 M calcium chloride, 500 μL 1 M MES, 500 μL 1 M potassium chloride, 50 mL deionized water, and pH was set to 5.5 with Tris-HCL. This solution can be frozen and stored for later. Next, solution B was prepared (0.45 g cellulase (EMD Millipore), 0.03 g pectolyase (Sigma-Aldrich), and 30 mL Solution A) and for each sample 7 mL of fresh Solution B was pipetted into 35-mm-diameter petri dishes. Creating bubbles was avoided when pipetting Solution B to avoid cell lysis at later stages in the protocol.

A 70 μm cell strainer was placed in each 35-mm-diameter petri dish. Approximately 1-2 mm of the root tip was cut to isolate the meristematic region of the root and put into the strainer in Solution B. The samples were incubated for 2 hours at 85 rpm at room temperature. The samples were regularly stirred. Next, Solution B and a small amount of cut roots were transferred to a 15 mL conical tube. The tubes were centrifuged for 6 min at 200 g. Supernatant was removed and the pellet was resuspended with 100 μL PIM. The resuspended solution was transferred to a 70 μm filter placed on top of a 50 ml conical tube. The 15 mL tube was rinsed with another 100 μL PIM, which was also transferred to the 70 μm filter. All the filtered liquid (also the liquid on the bottom of the filter) was transferred to a 40 μm filter placed on top of a 50 ml conical tube. The subsequent filtered liquid contains the protoplasts used for bioprinting.

Bioink and 3D-bioprinter. The protoplasts were printed with a CELLINK BIOX 3D bioprinter into an 8-well μ-slide, facilitating cell images at later time points. Various bioinks were used with 3D printers, as described further herein.

Staining and confocal imaging. Prior to image acquisition, cells were stained with 0.01% fluorescein diacetate (FDA, Sigma Aldrich) and calcofluor white (Sigma Aldrich). Image acquisition was performed on a Zeiss LSM 880 confocal microscope.

What is claimed is:

1. A matrix comprising a plurality of bioprinted plant cells, wherein the plurality of bioprinted plant cells are deposited in the matrix according to a pre-determined spatial pattern.

2. The matrix of claim 1, wherein the matrix is three-dimensional (3D) or two-dimensional (2D).

3. The matrix of claim 1 or claim 2, wherein the plurality of plant cells comprise at least one plant cell obtained from a meristematic region of a root and/or at least one plant stem cell.

4. The matrix of claim 1 or claim 2, wherein the plurality of plant cells comprise at least one stem cell obtained from a shoot and/or root stem cell niche.

5. The matrix of claim 1 or claim 2, wherein the plurality of plant cells comprise at least one cell obtained from a shoot and/or root apical meristem of a plant species.

6. The matrix of any of claims 1 to 5, wherein the plurality of plant cells are deposited according to a pre-determined temporal pattern.

7. The matrix of any of claims 1 to 6, wherein the plurality of plant cells are contained within a bioink composition.

8. The matrix of any of claims 1 to 7, wherein the matrix comprises at least one of agar, hydrogel, nanofiber, plant biomaterials, and any combinations thereof.

9. The matrix of any of claims 1 to 8, wherein the matrix further comprises at least one of a hormone(s), a phytohormone(s), a nutrient(s), an antibiotic(s), a prebiotic(s), a probiotic(s), a peptide(s), a polypeptide(s), a protein(s), a other growth factor(s), and any combinations thereof.

10. The matrix of any of claims 1 to 9, wherein the pre-determined spatial pattern induces the plurality of plant cells to form a callus.

11. The matrix of any of claims 1 to 10, wherein at least one of the plurality of plant cells is naturally occurring.

12. The matrix of any of claims 1 to 10, wherein at least one of the plurality of plant cells is genetically modified.

13. The matrix of any of claims 1 to 12, wherein the plurality of plant cells are deposited in the matrix according to at least one additional pre-determined metric selected from cell number, cell density, cell-type, cell arrangement, and bioink composition.

14. The matrix of any of claims 1 to 13, wherein the plurality of plant cells are deposited using a bioprinting device.

15. A plant callus formed from the plurality of bioprinted plant cells of claim 1.

16. A plant organ, organoid, or tissue formed or derived from the bioprinted plant cells of claim 1.

17. A system for bioprinting plant cells, the system comprising:
a plurality of plant cells;
a matrix whereupon the plurality of plant cells are deposited; and
a bioprinting device, wherein the bioprinting device deposits the plurality of plant cells in the matrix according to a set of pre-determined instructions.

18. The system of claim 17, wherein the bioprinting device comprises a processor component and a software component.

19. The system of claim 18, wherein the software component comprises the set of pre-determined instructions for bioprinting the plurality of plant cells, and wherein the processor component executes the set of pre-determined instructions.

20. The system of any of claims 17 to 19, wherein the set of pre-determined instructions comprises spatial and temporal information for depositing the plurality of plant cells in the matrix.

21. The system of claim 20, wherein the spatial and temporal information is based on a computational model comprising one or more of gene expression data, cell number, cell-type, rate of cell division, and any combinations thereof.

22. A method of producing a plant callus, the method comprising:
bioprinting a plurality of plant cells in a matrix; and
culturing the bioprinted plant cells in the matrix to induce the formation of a callus.

23. A plant callus formed using the method of claim 22.

24. A plant organ, organoid, or tissue formed using the method of claim 22.
